(19) United States
(12) Patent Application Publication
(10) Pub. No.: US 2011/0318405 A1
(43) Pub. Date: Dec. 29, 2011

(54) Chemical Combination and Method for Increasing Delivery of Coenzyme Q10

(76) Inventor: Charles Erwin, Wickenburg, AZ (US)

(21) Appl. No.: 13/215,114

(22) Filed: Aug. 22, 2011

Related U.S. Application Data
(63) Continuation-in-part of application No. 10/876,752, filed on Jun. 25, 2004, now Pat. No. 8,003,094.
(60) Provisional application No. 60/482,781, filed on Jun. 25, 2003.

(51) Int. Cl. A61K 9/00; A61K 31/122; A61K 38/43

(52) U.S. Cl. 424/448; 424/94.1; 514/689

(57) ABSTRACT

The present invention relates to a chemical combination and method for increasing delivery of Coenzyme Q10. The chemical combination comprises Coenzyme Q10 mixed with at least one chemical. The at least one chemical includes cyclic terpene containing essential oil(s) that permit unprecedented levels of Coenzyme Q10 to be made available for delivery and absorption, increasing bioavailability, as well as overcoming the previous limits. A transdermal patch including a layer containing Coenzyme Q10 is also provided.

### CROSS-REFERENCE TO RELATED APPLICATIONS

This application is a continuation-in-part of U.S. application Ser. No. 10/876,752 filed Jun. 25, 2004, which, in turn, claims the benefit of U.S. provisional Application No. 60/482,781 filed Jun. 25, 2003, the disclosures of which are incorporated in their entirety by reference herein.

### FIELD OF THE INVENTION

The present invention relates to a chemical combination, and more particularly, to a chemical combination that when combined with Coenzyme Q10, increases stable solubility, absorption, and efficacy of the Coenzyme Q10, and Coenzyme Q10 in its reduced or oxidized state by a user.

### BACKGROUND OF THE INVENTION

Reactions in the body produce chemicals called oxidants (free radicals). The oxidants damage cells and are generally thought to shorten one's life. As a way to protect the body from this damage, the body produces anti-oxidants such as Coenzyme Q10. Anti-oxidant nutrients terminate free radicals by donating electrons to the free radicals. The anti-oxidants become oxidized as part of the oxidation reduction reaction, however, the oxidized anti-oxidants do not contribute to the highest energy chain reactions as the free radicals do, which cause in excess of 80 diseases.

Found in a cell's mitochondria, Coenzyme Q10 is thought to be the primary limiting factor in the production of energy within each cell that the body uses to improve health. With this knowledge, researchers have attempted to incorporate Coenzyme Q10 into products with hopes of improving an individual's health through the production of more energy. However, studies have shown that while a small portion of Coenzyme Q10 pills get into the blood stream, even less Coenzyme Q10 is absorbed into the cell's mitochondria. Without being sufficiently recovered by the cell's mitochondria, Coenzyme Q10 products are much less effective than they would otherwise be. Thus, a need exists for a chemical combination that when combined with Coenzyme Q10, increases stable solubility, absorption efficacy, and the update of Coenzyme Q10, and Coenzyme Q10 in both the oxidized and reduced state by the cell's mitochondria.

### SUMMARY OF INVENTION

The present invention relates to a chemical combination for increasing delivery of Coenzyme Q10 to a cell. The chemical combination comprises Coenzyme Q10 mixed with at least one chemical. Through addition of the at least one chemical, the Coenzyme Q10 is able to overcome solubility problems and be absorbed more readily by a cell and a cell's mitochondria.

The at least one chemical comprises a solvent that functions as a carrier for the Coenzyme Q10. The solvent is selected from a group consisting of cetyl meristoleate, dl-alpha Tocopheryol acetate, dimethyl sulfoxide (DMSO), and d-limonene.

In another aspect, the at least one chemical further comprises at least one skin buffer for replacing oils stripped from a user's skin as a solvent passes through the user's skin. The at least one skin buffer is selected from a group consisting of dl-alpha Tocopheryol acetate, cetyl myristoleate, gamma linolenic acid, and conjugated linoleic acid.

In yet another aspect, the at least one chemical further comprises at least one anti-oxidant. The at least one anti-oxidant is selected from a group consisting of alpha lipoic acid, d-alpha Tocopheryol Succinate, Vinpocetin, Ergoloid mesylates, and Vitamin(s) A(s), B(s), C(s), D(s), E(s), F(s) and K(s).

Furthermore, the chemical combination is formed in a form suitable for a delivery method selected from a group consisting of inhalation, oral, intramuscular injection, intravenous (IV)-drip, lingual, gum, sub-lingual, nasal, anal, percutaneous transdermal absorption, and transdermal patch.

In another aspect, the chemical combination comprises:

1. the Coenzyme Q10 being approximately 1 to 85 percent by weight of the combination;
2. the solvent being approximately 10 to 90 percent by weight of the combination;
3. a first skin buffer being approximately 0 to 30 percent by weight of the combination;
4. a first anti-oxidant being approximately 0 to 20 percent by weight of the combination; and
5. a second skin buffer being approximately 0 to 30 percent by weight of the combination; and
6. a second anti-oxidant being approximately 0 to 20 percent by weight of the combination.

In a further aspect, the chemical combination comprises:

1. the Coenzyme Q10 being approximately 5 to 30 percent by weight of the combination;
2. the solvent being approximately 30 to 85 percent by weight of the combination;
3. a first skin buffer being approximately 1 to 25 percent by weight of the combination;
4. a first anti-oxidant being approximately 1 to 10 percent by weight of the combination; and
5. a second skin buffer being approximately 1 to 20 percent by weight of the combination; and
6. a second anti-oxidant being approximately 1 to 5 percent by weight of the combination.

In yet another aspect, the chemical combination comprises:

1. the Coenzyme Q10 being approximately 12 to 18 percent by weight of the combination;
2. the solvent being approximately 65 to 73 percent by weight of the combination;
3. a first skin buffer being approximately 1 to 20 percent by weight of the combination;
4. a first anti-oxidant being approximately 1 to 4 percent by weight of the combination; and
5. a second skin buffer being approximately 6 to 10 percent by weight of the combination; and
6. a second anti-oxidant being approximately 1 to 3 percent by weight of the combination.

In another aspect, the present invention is a method for applying a chemical combination. The method comprises acts of obtaining the chemical combination and administering the chemical combination to a user.

In another aspect, the present invention provides a transdermal patch comprising an adhesive a layer and a Co-enzyme Q10-containing composition. The Co-enzyme Q10-containing composition comprises a Co-enzyme Q10 and a solvent.

In another aspect, the present invention provides a transdermal patch comprising an adhesive a layer and a Co-enzyme Q10-containing composition. The Co-enzyme Q10-containing composition comprising Co-enzyme Q10, a solvent, and a component selected from the group consisting of antioxidants, vitamins, steroids, melatonin, minerals, muscle relaxants, anti-inflammatory compounds, and combinations thereof.

As can be appreciated by one in the art, the present invention is not limited to the chemical combination itself, but also includes a method for increasing delivery potential of Coenzyme Q10 by forming the chemical combination, and a method for transdermal delivery of Coenzyme Q10 by applying the chemical combination described herein.

### BRIEF DESCRIPTION OF THE DRAWINGS

Exemplary embodiments of the present invention will become more fully understood from the detailed description and the accompanying drawings, wherein:

FIG. 1A provides a schematic illustration of a single-layer drug-in-adhesive patch that includes a coenzyme Q10-containing transdermal composition;

FIG. 1B provides a schematic illustration of a multi-layer drug-in-adhesive patch that includes a coenzyme Q10-containing transdermal composition; and

FIG. 1C provides a schematic illustration of a reservoir patch that includes a coenzyme Q10-containing transdermal composition.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

The present invention relates to a chemical combination, and more particularly, to a chemical combination that when combined with Coenzyme Q10, increases stable solubility, absorption efficacy, and the uptake of oxidized Coenzyme Q10, and Coenzyme Q10 in its reduced state by the cell's mitochondria.

The following description, taken in conjunction with the referenced tables, is presented to enable one of ordinary skill in the art to make and use the invention. Various modifications will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein. Furthermore, it should be noted that unless explicitly stated otherwise, the numerical values in the tables included herein are illustrated qualitatively and without any specific scale, and are intended to generally present the concept of the present invention.

In order to provide a working frame of reference, first an introduction is provided to provide the reader with a brief understanding of the present invention. Second, a discussion of various aspects of the present invention is provided to give an understanding of the specific details.

### (1) Introduction

The present invention comprises a chemical combination for delivering Coenzyme Q10. The chemical combination utilizes a solvent to solve Coenzyme Q10 solubility, crystallization, re-crystallization, shelf life, rate of delivery, percentage delivered, efficacy and re-solublizing below body temperature which are problems encountered by nutritional formulators when sufficient Coenzyme Q10 is combined with a carrier(s) for bodily uptake. When applied, the chemical combination of the present invention increases delivery of Coenzyme Q10 to any entity using it, non-limiting examples of which include humans and animals.

The present invention also comprises new methods for delivering the product of the present invention. The delivery methods include inhalation, oral, intramuscular injection, intravenous (IV)-drip, lingual, gum, sub-lingual, nasal, anal and percutaneous transdermal absorption, and transdermal patch.

### (2) Discussion

The present invention is a novel and advanced nutritional product that utilizes a solvent to solve Coenzyme Q10 solubility problems with a faster rate of delivery and a higher percentage of the nutritional contents being sufficiently delivered. The solvent is any solvent that functions as a carrier of Coenzyme Q10 or Coenzyme Q10's synergistic compounds, non-limiting examples of which include cetyl meristoleate (CMO), dl-alpha Tocopheryol acetate, dimethyl sulfoxide, and d-limonene singly, and/or with other cyclic terpene containing essential oil(s), such as orange oil (which may contain 95% or more d-limonene). Non-limiting examples of d-limonene and/or cyclic terpene containing oils include Lavindin, Peppermint, Ginger, Camphor, Geranium, Orange, Lemon, Lavender, Tea Tree, and Rosemary. High dissolution and/or compounding with cyclic monoterpene containing essential oil(s) permits unprecedented levels of Coenzyme Q10 to be made available for delivery and absorption, increasing bioavailability, as well as overcoming the previous limits.

D-limonene combined with Coenzyme Q10 creates a new molecule, which takes in energy when combined with one another. D-limonene may also become a source of energy (fuel) within minutes, enabling the cells (e.g. heart cells) to produce more energy. Additionally, CMO allows d-limonene to be used on the skin, etc.

Bio-availability, taste, fragrance, tissue friendliness and synergistic considerations are addressed further by the addition of buffers and certain anti-oxidants. The term “Buffer” refers to a chemical that is added to a solution with properties sufficient for replacing oils stripped from a user's skin as solvents pass through the user's skin. The buffer may also be both a solvent and/or nutrient, as demonstrated by the present invention. Non-limiting examples of such buffers include dl-alpha Tocopheryol Acetate, lecithin, cetyl myristoleate, gamma linolenic acid, and conjugated linoleic acid. The skin buffers also function as anti inflammatory agents and auto-immune inhibitors. Non-limiting examples of such anti-oxidants include alpha lipoic acid, d-alpha Tocopheryol Succinate, Vinpocetin, Ergoloid mesylates, and Vitamins A(s), B(s), C(s), D(s), E(s), F(s) and K(s). The Vitamins would include all forms of each vitamin. For example, non-limiting examples of Vitamin B(s) would include the B3 Vitamins, such as niacinamide, niacin, inositol hexaniacinate, and methyl nicotinate.

The compounds also function as anti-inflammatory agents, anti auto-immune inhibitors, and solvents. Furthermore, anti-oxidants are synergistic with one another, especially vitamin(s) E and C, Coenzyme Q10, and alpha lipoic acid.

The present invention has shown remarkable improvements in cases of late stage congestive heart failure, severe gum disease and tooth abscess, multiple sclerosis, and the immune system functions. It is likely to favorably impact many other diseases, particularly chronic diseases. The present invention also solves other common problems associated with high concentration nutritional products, such as crystallization and re-crystallization, efficacy and resolublizing below body temperature. It is further noted that the present invention may also include formulas designed for other than percutaneous use, which may include high levels of dl-alpha Tocopheryol acetate, and/or lidocaine to buffer tissues and minimize pain associated with the injected material.

It is noted that d-limonene has “generally regarded as safe” (GRAS) status by the Federal Emergency Management Agency (FEMA), located at 500 C Street, SW Washington, D.C. 20472, since 1965 and is approved by the U.S. Food and Drug Administration (FDA), located at 5600 Fishers Lane, Rockville Md. 20857-0001, for food use.

In another refinement, the compositions set forth above include CLA (conjugated linoleic acid) and/or GLA (gamma linolenic acid). In a refinement, the GLA is present in an amount from about 0.0 percent to about 100 weight percent. In another refinement, the CLA is present in an amount from about 0 percent to about 100 weight percent of the total weight of the composition. In a refinement, the CLA is present in an amount from about 0.001 percent to about 99 weight percent. In another refinement, the GLA is present in an amount from about 0.001 percent to about 99 weight percent of the total weight of the composition. In another refinement, the CLA is present in an amount from about 0.5 percent to about 40 weight percent of the total weight of the composition. In another refinement, the GLA is present in an amount from about 0.5 percent to about 40 weight percent of the total weight of the composition. High amounts of CLA are useful for cancer detection by palpation while a highly purified GLA would be for non-healing ulcers or other ailments such as the femoro-patella syndrome, alone or in combination with the compositions set forth above.

In still another aspect of the present invention a composition suitable for transdermal delivery is provided. In a variation, the transdermal composition includes the chemical composition in related amounts set forth above. In particular, the chemical composition includes Coenzyme Q10 in an amount from approximately 1 to 85 percent by weight of the combination, a solvent in an amount from approximately 10 to 90 percent by weight of the combination, a first skin buffer being approximately 0 to 30 percent by weight of the combination, a first anti-oxidant being approximately 0 to 20 percent by weight of the combination, a second skin buffer being approximately 0 to 30 percent by weight of the combination; and a second anti-oxidant being approximately 0 to 20 percent by weight of the combination.

In another variation, the transdermal composition includes Coenzyme Q10 and a solvent as set forth above and a component selected from the group consisting of antioxidants, vitamins, steroids, melatonin, minerals, muscle relaxants, anti-inflammatory compounds, and combinations thereof. Examples of suitable solvents are set forth above. In particular, D Limonene and DMSO are the primary solvents while CMO is important for the absorption of the D Limonene fraction. Moreover, CMO remains in and on the surface of the skin the longest and promotes the last of the absorption of the products and is an important buffer for both the D Limonene and DMSO fractions. The products (i.e., components) can vary from 0 to 100% of the D Limonene fraction. The DMSO can vary from 0 to 100% of the DMSO fraction CMO is a part of the D-Limonene fraction. In a refinement, the products can vary from 10 to 60% of the D Limonene fraction and/or the DMSO can vary from 10 to 60% of the DMSO fraction CMO is a part of the D-Limonene fraction.

Examples of antioxidants include alpha lipoic acid, ascorbyl palmitate, ergoloid mesylates, and combinations thereof. In a refinement of the present variation, the transdermal composition includes alpha lipoic acid (a hydrogen bonded association which increases the delivery to the blood and mitochondria) present in an amount from about 10 to 90% of the total weight of the transdermal composition. In another refinement, the alpha lipoic acid is present in an amount from about 10 to 60% of weight of the transdermal composition. In still another refinement, the alpha lipoic acid is present in an amount of about 60% of the weight of the transdermal composition. In a further refinement, the transdermal composition includes ascorbyl palmitate typically in an amount from about 0.0001 to about 3.0% of the total weight of the transdermal composition. In another refinement, the transdermal composition includes ascorbyl palmitate typically in an amount of about 0.001% of the total weight of the transdermal composition. In a further refinement, the transdermal composition includes ergoloid mesylates typically in an amount from about 0 to about 15% of the total weight of the transdermal composition. In another refinement, the transdermal composition includes ergoloid mesylates typically in an amount of about 0.5% of the total weight of the transdermal composition. Examples of vitamins include the B12 vitamins (e.g., cyanocobalamine and methylcobalamine). In a refinement, the vitamins are present in an amount from about 0 to 99% of the total weight of the transdermal composition. In another refinement, the cyanocobalamine and methylcobalamine are each independently present in an amount from about 0.005 to about 33% of the total weight of the transdermal composition. In a further refinement, the cyanocobalamine and methylcobalamine are each independently present in an amount from about 0.001 to about 1% of the total weight of the transdermal composition. In still another refinement, the cyanocobalamine and methylcobalamine are each independently present in an amount of about 0.01% of the total weight of the transdermal composition. Examples of steroids include pregnenolone, testosterone, progesterone, antrostenodione, androstenodiol, 5-alpha hydroxy Laxogenin, DHEA, and the like. It should be appreciated that the coQ10 solutions acts as a buffer for ethyl alcohol and promotes the absorption of 5 alpha hydroxy Laxogenin. Each of these steroids are present either individually or in combination in an amount from about 0 to 30 weight percent of the total weight of the composition. In a refinement, the transdermal composition further includes pregnenolone in an amount from about 0 to 12.6% of the total weight of the transdermal composition. In another refinement, the pregnenolone is present in an amount of about 4.2% of the total weight of the transdermal composition. In a refinement, the transdermal composition further includes progesterone in an amount from about 5 to 15% of the total weight of the transdermal composition. In another refinement, the progesterone is present in an amount of about 7.5% of the total weight of the transdermal composition. In a refinement, the transdermal composition further includes androstenodione and/or androstenodiol in an amount from about 5 to 15% of the total weight of the transdermal composition. In another refinement, the androstenodione is present in an amount of about 8% of the total weight of the transdermal composition. In a refinement, the transdermal composition further includes testosterone in an amount from about 5 to 15% of the total weight of the transdermal composition. In another refinement, the testosterone is present in an amount of about 8% of the total weight of the transdermal composition. In a refinement, the transdermal composition further includes DHEA in an amount from about 5 to 15% of the total weight of the transdermal composition. In another refinement, the DHEA is present in an amount of about 8.4% of the total weight of the transdermal composition. In a refinement, the transdermal composition further includes strontium (in the form of strontium chloride hexahydrate, SrCl₂(H₂O)₆ in an amount from about 0 to 30% of the total weight of the transdermal composition. Strontium chloride hexahydrate is usually added if DMSO is present which acts as a buffer and also increases the solubility of magnesium chloride hexahydrate in the DMSO solutions. In another refinement, the testosterone is present in an amount of about 11% of the total weight of the transdermal composition. In a refinement, the transdermal composition further includes melatonin in an amount from about 0 to 12% of the total weight of the transdermal composition. In another refinement, the melatonin is present in an amount of about 4.2% of the total weight of the transdermal composition. In a refinement, the transdermal composition further includes magnesium chloride (e.g., magnesium chloride hexahydrate) in an amount from about 0 to 40% of the total weight of the transdermal composition. Magnesium chloride hexahydrate is usually added if DMSO is present which acts as a buffer. In another refinement, the magnesium chloride is present in an amount of about 4.2% of the total weight of the transdermal composition. In a refinement, the transdermal composition further includes lobelia seed extract in an amount from about 0 to 20% of the total weight of the transdermal composition. In another refinement, the lobelia seed extract is present in an amount of about 5% of the total weight of the transdermal composition. The transdermal composition may also include GLA and CLA in amounts as set forth above.

In still another variation, the compositions set forth above are formed into a D Limonene fraction and a DMSO soluble fraction and an ethyl alcohol fraction. That is, the components set forth above (e.g., coQ10, buffers, vitamins, steroids, anti-oxidants) may be dissolved into one or more of these solvents depending on their solubilities. In a refinement, a combined composition includes 0 to 100 weight % of the D Limonene fraction, 0 to 100 weight % of the DMSO fraction, and 0 to 100 weight percent of the ethyl alcohol fraction. In another refinement, a combined composition includes 5 to 95 weight % of the D Limonene fraction and/or 5 to 95 weight % of the DMSO fraction, and/or 5 to 95 weight % of the ethyl alcohol fraction. Examples of DMSO soluble components include magnesium chloride hexahydrate, SrCl₂(H₂O)₆, alpha lipoid acid, ergoloid mesylates, cyanocobalamine and methylcobalamine, pregnenolone, testosterone, progesterone, antrostenodione, androstenodiol, and combinations thereof. Examples of D limonene soluble components include, but are not limited to, Vitamin A's, C's as Ascorbyl palmitate, D as D3 cholecalciferol, E's as d-alpha tocopherol succinate, Vinpocetin and Coenxyme Q10. Examples of components soluble in ethyl alcohol include, but are not limited to, 5-alpha hydroxy Laxogenin, and other components of the coQ10 solutions. It should also be pointed out that the ethanol fraction needs to be heated and stirred (e.g., for several hours) at a medium speed and a temperature of about 47° C. so that the coQ10 and the other components set forth above (e.g., buffers, vitamins, steroids, anti-oxidants) cross into the ethanol and warmed to 40-45 degrees when applied to a subjects skin.

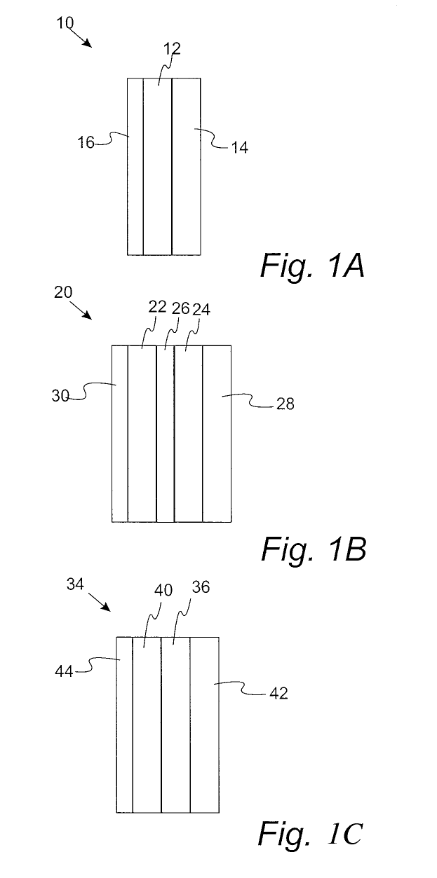

In still another aspect, a transdermal patch for delivering the transdermal composition set forth above is provided. In a variation, the transdermal patch includes a layer containing the transdermal composition. In a refinement, the transdermal patch is a single-layer drug-in-adhesive patch as set forth in FIG. 1A. In FIG. 1A, the transdermal patch 10 includes adhesive layer 12 which includes the transdermal composition. The adhesive layer adheres the patch to skin. Transdermal patch 10 also includes backing 14 and temporary liner 16 which is removed prior to application to the skin.

In another refinement, the transdermal patch is a multi-layer drug-in-adhesive patch as set forth in FIG. 1B. In this variation, transdermal patch 20 includes multiple adhesive layers such as adhesive layers 22, 24 which include the transdermal composition. In a further refinement, one of adhesive layers 22, 24 is for immediate release while the other is for delayed release. In another refinement, transdermal patch 20 includes a membrane 26 between adhesive layers 22, 24. Transdermal patch 10 also includes backing 28 and temporary liner 30 which is removed prior to application to the skin.

In another refinement, the transdermal patch is a drug in reservoir patch as set forth in FIG. 1C. In this refinement, transdermal patch 34 includes reservoir 36 which includes the transdermal composition. Reservoir 36 is a compartment that holds the transdermal composition. Transdermal patch 34 includes adhesive layer 40 for adhering to skin and backing 28 and temporary liner 42 which is removed prior to application to the skin.

The following example is provided for a further understanding of the invention, however, the invention is not to be construed as limited thereto. It can be appreciated by one in the art that the percentages associated with each ingredient in the chemical combination can be changed and will fluctuate according to the particular application.

### Example

This example is directed to a specific chemical combination according to the present invention. It is noted that concentrations of at least 1% Coenzyme Q10 and above are also possible. In this example, the 15% concentration is chosen for illustrative purposes only.

The identification and amounts of ingredients are as follows:

| | APPROXIMATE |
|---|---|
| | AMOUNT |
| INGREDIENT | (% by weight) |
| Coenzyme Q10. | 15 |
| A solvent, such as d-limonene | 70 |
| A first skin buffer, such as cetyl myristoleate | 8 |
| An anti-oxidant, such as Vinpocetin powder | 2 |
| A second skin buffer, such as dl-alpha Tocopheryol | 3 |
| Acetate | |
| A second anti-oxidant, such as d-alpha Tocopheryol | 2 |
| Succinate | |

The above formulation is prepared by mixing each of the ingredients in a vessel. The size of the vessel depends upon the amount of the solution desired.

In addition, if the solution solidifies, then it may be warmed to any applicable temperature allowing the solution to resolubilize, such as to 37 degrees centigrade.

The present invention also comprises a method for increasing delivery of a chemical solution and/or Coenzyme Q10 to a cell and to a cell's mitochondria. The method comprises an act of forming a chemical mixture and/or compound comprised of Coenzyme Q10 and a combination of chemicals.

For further illustration, the following is a non-limiting example of a process of creating a chemical combination according to the present invention. In this non-limiting example, one kilogram of a liquid product with a 18% concentration of Coenzyme Q10 is produced, combining the ingredients listed below in the following sequence, amounts and manner:
A. Mixing 180 grams of Coenzyme Q10 powder with 720 grams of d-limonene in a flask;
B. continuously stirring while warming the combination to approximately 37 degrees centigrade;
C. adding 10 grams of Vinpocetin powder;
D. adding 80 grams of cetyl myristoleate;
E. adding 10 grams of d-alpha Tocopheryol Succinate; and
F. sealing the final combination in a flask to minimize loss of volatile components.

For further illustration, the following is a non-limiting example of a process of creating a chemical combination according to the present invention. In this non-limiting example, 1 kg of a liquid product with a 10% concentration of Lobelia seed extract is produced combining the ingredients listed below in the following sequence amount and manner:
Mixing 698 gm of DMSO with lobelia seeds extract in a flask,
Continuously stirring while warming the combination to approximately to 45 degree centigrade
Adding 200 gm of alpha lipoic acid
Adding 10 gm of pregnenolone
Adding 20 gm of progesterone
Adding 20 gm of DHEA
Adding 20 gm of Mg,Cl 2 hexahydrate
Adding 10 gm of Strontium chloride hexahydrate
Adding 10 gm of ergoloid mesylates
Adding 1 gm each of cyanocobalamine and methylcobalamine (B12)
Sealing the final combination in a flask to minimize loss of volatile components

The following is another non-limiting example of a process of creating a chemical combination according to the present invention. This combination of ingredients is useful for kidney disease, heart disease, muscle atrophy and bone repair and other inflammatory conditions and/or chronic diseases. In this non-limiting example, 1.2 kg of a liquid product with a 2% concentration of 5 alpha hydroxyl-Laxogenin in Ethyl alcohol is produced combining the ingredients listed below in the following sequence amount and manner:

Mixing 12 gm of 5 alpha hydroxyl Laxogenin with 600 gm of Ethyl alcohol in a flask,

Continuously stirring while warming the combination to approximately 45 degrees centigrade

Adding 588 gm of coQ10/d-Limonene solution

Continuously stifling the combination at 45 degrees centigrade

Ceiling the final solution in a flask to minimize loss of volatile components:

It can be appreciated by one skilled in the art that the aforementioned process can be altered and manipulated to achieve any desirable result. For example, should one desire a different concentration of Coenzyme Q10, the amounts included in the combination would be changed. Additionally, the steps by which the chemicals are added can also be altered to achieve the desired result. Furthermore, certain buffers, anti-oxidants, and solvents can be omitted or changed in various combinations thereto.

In another aspect, the chemical mixture of the present invention is formed in such a way that it allows for effective delivery, non-limiting examples of such delivery methods include inhalation, oral, intramuscular injection, IV-drip, lingual, gum, sub-lingual, nasal, anal, percutaneous transdermal absorption, and transdermal patch.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A transdermal patch comprising:
an adhesive layer; and
a composition comprising:
co-enzyme Q10; and
a solvent.

2. The transdermal patch of claim 1 further comprising a first skin buffer, a first anti-oxidant, a second skin buffer, and a second anti-oxidant.

3. The transdermal patch of claim 2 wherein
the coenzyme Q10 is present in an amount from 5 to 30 percent by weight of the total weight of the composition;
the solvent is present in an amount from 30 to 85 percent by weight of the total weight of the composition wherein the solvent is selected from the group consisting of cetyl myristoleate, dimethyl sulfoxide, d-limonene, the essential oil lavindin, the essential oils of peppermint, ginger, camphor, geranium, orange, tea tree, lavender, cyclic terpenes containing essential oils, and mixtures thereof;
the first skin buffer is present in an amount from 1 to 25 percent by weight of the total weight of the composition wherein the first skin buffer is selected from the group consisting of dl-alpha Tocopheryol acetate, cetyl myristoleate, gamma linolenic acid, conjugated linolenic acid, d-alpha Tocopherol, lecithin, lidocaine, and mixtures thereof;
the first anti-oxidant is present in an amount from 1 to 10 percent by weight of the total weight of the composition;
the second skin buffer is present in an amount from 1 to 20 percent by weight of the total weight of the composition; and
the second anti-oxidant is present in an amount of 1 to 5 percent by weight of the total weight of the composition.

4. The transdermal patch of claim 2 wherein the second skin buffer is selected from the group consisting of dl-alpha Tocopheryol acetate, cetyl myristoleate, gamma linolenic acid, conjugated linoleic acid, d-alpha Tocopherol, lecithin, lidocaine, and mixtures thereof.

5. The transdermal patch of claim 2 wherein the solvent is d-limonene.

6. The transdermal patch of claim 2 wherein the first skin buffer is cetyl myristoleate and the second skin buffer is dl-alpha Tocopheryol acetate.

7. The transdermal patch of claim 2 wherein the first anti-oxidant is alpha lipoic acid and the second anti-oxidant is d-alpha Tocopheryol Succinate.

8. The transdermal patch of claim 2 wherein the first and second anti-oxidants are independently selected from the group consisting of alpha lipoic acid; d-alpha Tocopheryol Succinate; Vinpocetin; Ergoloid Mesylates; and Vitamin(s) A(s), C(s), D(s), E(s), and K(s); and mixtures thereof.

9. The transdermal patch of claim 1 wherein the composition comprises a fraction selected from the group consisting of a D Limonene fraction, a DMSO soluble fraction, an ethyl alcohol fraction, and combinations thereof.

10. A transdermal patch comprising:
an adhesive layer; and
a composition comprising:
co-enzyme Q10;
a solvent; and
a component selected from the group consisting of antioxidants, vitamins, steroids, melatonin, minerals, muscle relaxants, anti-inflammatory compounds, and combinations thereof.

11. The transdermal patch of claim 10 wherein the transdermal composition includes alpha lipoic acid in an amount from about 10 to 90% of the total weight of the transdermal composition.

12. The transdermal patch of claim 11 wherein the transdermal composition includes ascorbyl palmitate in an amount from about 0.0001 to about 0.003% of the total weight of the transdermal composition.

13. The transdermal patch of claim 12 wherein the transdermal composition includes ergoloid mesylates typically in an amount from about 0.01 to about 1% of the total weight of the transdermal composition.

14. The transdermal patch of claim 13 wherein the transdermal composition includes cyanocobalamine and methylcobalamine.

15. The transdermal patch of claim 14 wherein the transdermal composition includes a component selected from pregnenolone, testosterone, progesterone, 5-alpha hydroxy Laxogenin, antrostenodione, androstenodiol, and combinations thereof.

16. The transdermal patch of claim 15 wherein the transdermal composition includes a component selected from the group consisting of DHEA, strontium chloride hexahydrate, magnesium chloride hexahydrate, lobelia seed extract and combinations thereof.
